(19) United States
(12) Patent Application Publication
(10) Pub. No.: US 2022/0265575 A1
(43) Pub. Date: Aug. 25, 2022

(54) NON-STAINING CURCUMINOID COMPOSITION

(71) Applicant: INVENTIA HEALTHCARE LIMITED, Mumbai (IN)

(72) Inventors: Vishal SHAH, Mumbai (IN); Rajat SHAH, Mumbai (IN); Manoj TIWARI, Thane (IN); Shajahan ABDUL, Madurai (IN)

(21) Appl. No.: 17/753,015

(22) PCT Filed: Aug. 19, 2020
(86) PCT No.: PCT/IB2020/057789

§ 371 Date: Feb. 15, 2022

(30) Foreign Application Priority Data
Aug. 22, 2019 (IN) 201921033852

(51) Int. Cl. A61K 31/12; A61K 9/16

(52) U.S. Cl. CPC A61K 31/12; A61K 9/1617; A61K 9/1664

(57) ABSTRACT

The invention describes a non-staining composition comprising high content of curcuminoids in granular form, which is substantially free of excipients and is comprised of at least about 94% of curcuminoids. The non-staining composition may be comprised of 0 to 5% by weight of excipients, which are used as processing aids. The non-staining curcuminoid composition does not cause staining and dusting of the equipment and the contact surfaces, while formulating into finished dosage forms. The invention also relates to solvent-free process for preparation of non-staining granular composition; wherein the curcuminoids may be subjected to granulation process at specific temperature conditions. The granular composition is free flowing and retains original yellowish orange colour of the curcuminoids along with its crystalline nature. The solvent-free process for non-staining granular curcuminoid composition is simple, economical and environment friendly, thus making it industrially useful and applicable for formulating finished dosage forms.

### FIELD OF THE INVENTION

The present invention relates to a non-staining composition comprising high content of curcuminoids in granular form, and process for the preparation thereof. The composition is substantially free of excipients and is comprised of at least about 94% of curcuminoids. The non-staining composition may be comprised of about 0 to 5% by weight of excipients, which are used as processing aids, while preparing the composition. The invention specifically relates to non-staining curcuminoid composition, which retains original colour as well as crystallinity of curcuminoids, but does not cause staining and dusting on the equipment and the contact surfaces, while formulating into finished dosage forms. The invention also relates to solvent-free process for preparation of non-staining, granular and free flowing composition with high content of curcuminoids; wherein the curcuminoids may be subjected to granulation process at specific temperature conditions. The process is simple, economical and environment friendly as well as industrially applicable. The non-staining curcuminoid composition retains original properties of curcuminoids and is suitable for subjecting to various formulation related operations, without causing any dusting and staining like unformulated curcuminoids, thus making the formulation process clean, non-expensive and non-time consuming.

### BACKGROUND

Turmeric is the rhizome of the plant Curcuma longa Linn, a tropical herb of the Zingiberaceae family and native to southern Asia. It is primarily consumed in the form of powdered rhizomes mainly for colouring because of its yellowish orange colour and its associated medicinal properties. It also imparts the characteristic flavour and preserves the freshness of the product. The rhizomes contain curcuminoids (3-6% of curcumin, desmethoxycurcumin, and bisdemethoxycurcumin), which are natural polyphenols which are responsible for the yellow colour. The curcuminoid pigments and volatile oils, which are the major secondary metabolites of turmeric, have been shown to be largely responsible for the pharmacological activities of turmeric powder, extracts and oleoresins. In Asian countries, fresh and dry turmeric rhizomes as well as ground turmeric is used for vegetable and meat dishes and soup-like products. Turmeric is commercially available as dried rhizome powder, curcuminoids, essential oils and oleoresin.

Curcuminoids have demonstrated antiparasitic, antispasmodic, and anti-inflammatory effects, as well as potential anticancer activity. The rhizome has been also used in food and Ayurvedic medicines since thousands of years for its medicinal properties. Curcuminoids is also known for its use in liver disorders, and wide variety of pulmonary, gastrointestinal and skin diseases. It keeps skin soft and smooth, makes it glow, tackles acne and helps treat hyper-pigmentation. Turmeric paste is also prescribed in Indian medicine for various skincare issues, like puffiness, wrinkles and eczema. Besides this, it is used for cuts and burns due to its antiseptic effect and ability to promote healing. In addition, it can help to decrease muscle soreness after an intense workout.

Even though there has been an increasing interest in turmeric due to its medicinal properties over the past few years, handling or processing of this product in regular powder form is a big problem due to its staining and dusting properties. Curcuminoids stain the equipment and contact surfaces, while handling it or formulating in dosage forms, and therefore make the production process troublesome and time consuming. Curcuminoids also cause dusting in the formulation area and therefore once curcuminoids are processed, the entire plant needs complete change over cleaning, which is very often done using organic solvents, thus involving considerable cost, time and therefore affecting the production efficiency.

Prior art patent applications have tried to tackle this problem in variety of ways, but the efforts are mainly focused on either coating of curcumin for masking the colour using organic solvents and polymer; or its chemical transformation through various catalytic or other hydrogenation reactions.

US patent application 20180042858 (Akay Flavors and Aromatics Pvt Ltd) relates to non-bleeding curcumin comprising curcumin and a coating of polymer, wherein the polymer is selected from the group consisting of a biopolymer, synthetic polymer or a mixture thereof. In these compositions, the biopolymer is selected from the group consisting of cellulose, shellac, galactomannans, glucomannans, arabinoxylans, natural gums, proteins or a mixture thereof and the synthetic polymers is selected from the group consisting of polyvinyl alcohol, polylactic acids or a mixture thereof. The compositions eliminate the problem of colour bleeding and colour leaching during industrial processing of curcumin and makes use of water-based coating process for preparation.

Indian patent application 1853/DEL/2008 (Ind Swift Limited) relates to non-staining curcumin topical formulation comprising turmeric or extracts thereof such as curcumin or curcuminoids along with excipients which include a specialized base solution and a specialized solubilizer in specific concentrations. Polyoxyl hydrogenated castor oil derivative is used as specialized solubilizer; while propylene glycol, polyethylene glycol and glycerol are used as specialized solubilizer.

Indian patent application IN 201402079 (COUNCIL OF SCIENTIFIC AND INDUSTRIAL RESEARCH) discloses a process for the preparation of glycosylated tetrahydrocurcumin, which is a major colourless metabolite of curcuminoids isolated from Curcuma longa. The process was comprised of glycosylation process and use of a phase transfer catalyst. Tetrahydrocurcumin-di-β-glycoside was synthesized, and its biological and pharmacological activity was evaluated. The accelerated wound-healing process with non-toxic, non-irritant and non-staining properties render this multi-functional compound tetrahydrocurcumin-di-β-glucoside as a useful ingredient in achromatic food and cosmetic applications.

U.S. Pat. No. 8,568,802 (Laila Impex) relates to a process for producing an enriched fraction of tetrahydoxycurcumin containing colourless tetrahydro-derivatives by the process of demethylation of natural curcumins, obtained, in turn, from the organic solvent extract of turmeric from Curcuma species. The said enriched fraction of tetrahydroxycurcumin is subjected to hydrogenation to get colourless tetrahydrotetrahydroxycurcumin enriched fraction. The enriched fractions of tetrahydroxycurcumin and colourless tetrahydroxycurcumin exhibit potent antioxidative action and reduces inflammation.

U.S. Pat. No. 7,763,289 (JOAI'S Products LLC) describes a topical skin care product containing therapeutic concentrations of turmeric and other constituents which is colourless upon application to the skin and process for the preparation. The process of preparing the composition is comprised of mixing a turmeric extract with a berry extract in a ratio from 1:1 part to 1:20 part (v/v) and maintaining pH between 4.0 and 6.0.

US patent application 20170258743 (Aurea Biolabs Pvt Ltd) relates to a process for preparation of white curcumin, the process comprising the steps of treating curcuminoids with the organic solvent to form a reaction mixture and adding palladium carbon as catalyst to the reaction mixture; subjecting the reaction mixture to hydrogenation in the presence of hydrogen gas; filtering and washing the filtrate with ethyl acetate; distilling the ethyl acetate to obtain a crude mass of white curcumin and finally stirring with purified water to obtain a solid precipitate of white curcumin.

JP2009114176 (Microcel KK) relates to process wherein fat-soluble components contained in turmeric rhizomes are extracted with an organic solvent, and the extract is subsequently treated with a bleaching agent to remove curcumin. Tetrahydrodemethoxy diferuloylmethanes, which are colourless endogenous metabolites of curcumin, are added to the extract from which curcumin has been removed. The obtained liquid is added to the carrier oil to produce a massage oil or added to an aqueous base comprising deep ocean water taken from a plurality of depths to produce a cosmetic.

U.S. Pat. No. 6,653,327 (Sami Labs) describes a mixture of tetrahydrocurcuminoids which is substantially colourless and can be used through topical administration for regulating random intracellular protein crosslinking, optimizing cell electric potential, treating a premalignant state or a malignancy, and reducing “age spots” due to lipofuscin formation.

Prior art references mainly focus on coating of curcuminoids by synthetic or natural polymers using organic solvents. Some of the patent applications relate to chemical modification of curcuminoids through demethylation or hydrogenation, for overcoming problem of colour explosion and dusting during industrial processing of curcuminoids. However, this results into chemical transformation of curcuminoids along with change in its natural bright yellowish orange colour, which may not be desired for application in nutraceutical and food industry. Further the coating of curcuminoids by polymers for colour masking depends on percentage of coating applied on the active. Higher amount of coating effectively lowers down the content of curcuminoids in the final formula, rendering the final formulation less effective for various industrial applications.

Thus, there is an unmet need to find out alternate ways to overcome the staining property of curcuminoid, without disturbing or disrupting its chemical identity and physical characteristics as well as colour, thus making the product acceptable and suitable for foods, cosmetics and nutraceuticals applications.

### OBJECTIVES

Main objective of this invention is to provide a non-staining granular composition with high content of curcuminoids, which is substantially free of any excipient.

Another objective of the invention is to provide non-staining curcuminoid composition, which is comprised of at least 94% curcuminoids.

One more objective of the invention is to provide a granular curcuminoid composition which does not cause staining and/or dusting on the equipment and contact surfaces, while formulating in final dosage forms.

One more objective of the invention is to provide a non-staining granular curcuminoid composition, which may contain 0 to 5% by weight of excipients, used as processing aid, which facilitate preparation of the composition by solvent free granulation process.

Still one more objective of the invention is to provide a non-staining granular composition, in which curcuminoids retain their original yellowish orange colour and crystallinity as of unformulated curcuminoids, thus making the product most suitable for converting into desired dosage forms.

One important objective of the invention is to provide a solvent-free process for preparation of non-staining granular curcuminoid compositions wherein the active is processed by granulation using heat treatment varying from 50 to 200° C.

One objective of the present invention is to provide non-staining granular curcuminoid composition, wherein the size of granules ranges from 50 to 1000 microns.

One important objective of the instant invention is to provide non-staining curcuminoids composition, which prevents staining and dusting on equipment and other surfaces in contact, while converting the granules into finished dosage forms.

One important objective of the present invention is to provide non-staining curcuminoid composition, which is convenient to formulate into finished dosage forms, thus making the formulation process clean, non-expensive and non-time consuming.

### SUMMARY

Present invention relates to non-staining composition with high content of curcuminoids in granular form, which is substantially free of excipients and a solvent-free process for preparation thereof. More specifically, the composition is comprised of at least about 94% curcuminoids and it retains original yellowish orange colour and crystallinity pattern of curcuminoids.

The invention also relates to solvent-free granulation process for preparation of non-staining curcuminoid granular compositions, using heat treatment.

The granular composition prevents staining of equipment, body parts and all other surfaces which come in contact, while converting the granules into finished dosage forms. Non-staining curcuminoid composition also prevents dusting on the equipment and formulation area while carrying out various preparation and formulation steps. The granular curcuminoid composition avoids time and cost consuming cleaning operations while carrying out the batches of curcuminoids in plant along with other products. The non-staining composition, as described herein, thus makes the formulation activity clean, non-expensive and non-time consuming.

### DETAILED DESCRIPTION

It has surprisingly been found that granular composition of the present invention comprising high content of curcuminoids, which is substantially free of excipients, exhibits non-staining property. The composition is comprised of at least about 94% of curcuminoids and retains original colour as well as crystalline pattern like that of curcuminoids. The granular curcuminoid composition prevents staining of equipment, body parts and all other surfaces in contact, while processing the granules for converting into finished dosage forms. The composition is thus useful for further processing of the granules in desired dosage forms, as the granules do not cause any dusting on equipment and formulation area which otherwise happens due to unformulated plain curcuminoids.

The present invention uses curcuminoids, purchased in the form of extract from the external vendor. The extract is obtained from Curcuma species of the Zingiberaceae (ginger) family, and contains specific percentage content of curcuminoids.

The term ‘curcuminoid extract’ is used herein to mean the extract of roots, tubers or rhizomes of the Curcuma species of the Zingiberaceae (ginger) family including but not limited to species like Curcuma longa, Curcuma amada, Curcuma angustifolra, Curcuma caesia, Curcuma zedoaria, Curcuma caulina, Curcuma aromatica, Curcuma wenyujin, Curcuma xanfhorrhiza, Curcuma phaeocaulis, Curcuma leucorrhiza and Curcuma kwangsiensis.

The term ‘curcuminoid’ is used herein to mean curcumin (diferuloylmethane), desmethoxycurcumin, or bisdesmethoxycurcumin, including their polymorphs, hydrates, solvates, derivatives, and mixtures thereof.

The terminology ‘non-staining’ as used herein, is the property exhibited by granular curcuminoid composition, as described herein, by virtue of which, the composition does not stain or colour any equipment and contact surfaces, which are used to handle granules during formulation into desired dosage forms. The composition also does not cause dusting on the formulation area and equipment, while carrying out different processes like weighing, sifting, mixing or during filling in the capsules or compression in tablet dosage forms. Non-staining composition is obtained from curcuminoids, which has characteristic yellowish orange colour. Even though this original colour as well as crystallinity is retained in non-staining granular composition, it does not significantly stain or colour any of the contact surfaces or formulation equipment, while formulating into finished dosage forms. The non-staining composition does not even significantly stain the hands while handling the granules. Thus even though the composition is prepared from curcuminoids, which have characteristic yellowish orange colour, the composition exhibits non-coloring characteristic, in its granular form.

The terminology ‘substantially free of excipients’ as used herein means the composition may contain only about 0 to 5% of the excipients by weight of the composition, which may be used as processing aid, while preparation of non-staining composition in granular form. Thus as per one embodiment, the composition may be substantially free of excipients. As per one more embodiment, the composition may contain 1 to 5% of excipients by weight of the composition, which may be selected from the group of diluent, filler, carrier, stabilizer, antioxidant, disintegrant, lubricant, antiglidant, anti-tacking agent, oil vehicle and the like, which are commonly used in pharmaceutical, nutraceutical, food and cosmetics industry. According to one important embodiment, the processing aid may be selected from the group of lipids, fats, fatty acids, fatty alcohols, fatty acid esters, waxes, oils and the combination thereof.

The processing aid may facilitate preparation of the composition in free-flowing granular form, which have non-staining property. The processing aid may be required to convert the extract comprising high content of curcuminoids in the form of granules, using process of heat treatment and granulation.

The terminology ‘high content of curcuminoids’ as used herein means the composition comprised of at least about 94% of curcuminoids, calculated as total curcuminoids. Thus these compositions may contain about 94 to 100% of curcuminoids in granular form and the composition may be substantially free of excipients. Thus the composition may be free of any excipients or may contain only about 1 to 5% by weight of processing aids, thus resulting into very high content of curcuminoids in the non-staining granular composition. The granules having non-staining property can be used by formulators for converting into desired dosage forms as per the requirement of active content.

The terminology ‘yellowish orange’ as used herein refers to wide colour range of curcuminoids obtained from turmeric extract, wherein the colour may vary from different shades of yellow to red to orange. The colour shade of curcuminoids may vary depending upon source, processing conditions, storage conditions, but the spectrum of color shade may vary from bright yellow, dull yellow, yellowish orange to light orange or reddish orange or dark orange, and the possible shades of curcuminoids lying between this colour range. The granular non-staining composition as described herein retains the original color shade of plain unformulated curcuminoids; but it exhibits the property in the granular composition as described herein, wherein it does not significantly stain the equipment and contact surfaces, while processing into final formulations.

The terminology ‘solvent-free process’ as used herein refers to the process for preparation of non-staining granular composition of the invention, which is comprised of high content of curcuminoids. The process does not make use of any organic solvents while preparing the granules, thus rendering the process simple, cost and time effective as well as environment friendly. The process makes use of granulation process using heat treatment and suitable granulator equipped for application of heat. Solvent-free process, as used herein also provides the advantage of getting non-staining granular product, which is free from traces of any residual organic solvent, thus making the product safe for consumption for various application in nutraceutical, food and cosmetic industry.

According to one embodiment of the present invention, the composition uses a curcuminoid extract containing at least about 94% by weight of one or more curcuminoids.

As per one more embodiment of the invention, the non-staining composition may be comprised of high content of curcuminoids, wherein the granules may contain about 94% to 100% of curcuminoids, calculated as total curcuminoids.

According to one more embodiment of the invention, the composition is free flowing and granular in nature and the granule size may range from 50 to 1500 microns. More preferably the size of granules may range from 100 to 1000 microns, most preferably it may range from 200 to 800 microns.

According to another embodiment of the invention, the granular composition may be comprised of curcuminoids which are substantially crystalline in nature. The granular composition retains the original crystallinity of curcuminoids even after converting in the granular composition.

Curcuminoids have their characteristic yellowish orange colour, which is mainly due to its polyphenol content. The composition as described herein, retains its characteristic yellowish orange colour after processing into the granules. The composition exhibits non-staining property, because of which it does not stain the equipment, contact surfaces and even the formulation area while converting into finished dosage forms.

As per one important embodiment of the invention, the composition is in the form of non-staining granules, which may prevent dusting of curcuminoids while handling and processing it during weighing, mixing, granulation, compression, filling and so on, for converting in finished dosage forms like tablets, capsules and the like.

As per one more embodiment of the current invention, the composition may be substantially free of excipients. The composition may contain about 0 to 5% by weight of processing aids, which may facilitate process for preparation of the composition in its granular form.

According to another embodiment, the composition may be comprised of 100% by weight of curcuminoids, and thus do not contain any processing aid in the granules.

As per one more embodiment, the composition may be comprised of 1 to 5% by weight of processing aids, thus the composition may be comprised of very high content of curcuminoids, ranging from 95 to 100% in the granular composition.

According to one important embodiment of the invention, the processing aid may be selected from the group of lipids, fats, fatty acids, fatty alcohols, fatty acid esters, waxes, oils and the combination thereof.

As per one more embodiment, the processing aid may be selected from, but not limited to, saturated fatty acids having 12 to 28 carbons, such as stearic acid, fatty alcohols having from 16 to 44 carbons, cetyl alcohol, pegylated fatty acids, glycerol fatty acid esters, monoglycerides, diglycerides, triglycerides, derivatives of mono-diglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, polyglycerol esters, glycerolipid, phosphatic acid, phosphatidylethanolamine, phosphatidylcholine, phosphatidylserine, phosphatidylinositol or other glycerophospholipids, ceramide, sphingolipid, sterol, fat-soluble vitamin, prenol, saccharolipid, polyketide, their salts and esters and the combination thereof.

Processing aid may also be selected from, but not limited to, beeswax, candelilla wax, carnauba wax, spermaceti, paraffin wax, synthetic waxes and the combination thereof.

According to one embodiment of the invention, the compositions may be prepared by solvent-free process using suitable granulation process, selected from dry granulation, melt granulation, extrusion, coacervation, congealing, steam granulation, freeze granulation, foam granulation and the like.

According to one embodiment of the invention, the granular composition may be prepared by single step or multi-step granulation, using various suitable granulation equipment such as rapid mixer granulator, extrusion spheronizer, hot melt extruder, roller compactor, fluid bed granulator, generally used in food, nutraceutical or pharmaceutical industry.

According to another embodiment, the composition may be prepared by one step continuous granulation process by processing the active in twin screw processor. Curcuminoid extract may be subjected to continuous granulation process, wherein the input material is continuously fed into the granulator, while the output (granular non-staining composition) is taken out in one-step process.

As per one embodiment, the solvent-free process as described herein may be comprised of granulating curcuminoids by heat treatment to get granular mass. The mass may be optionally mixed with about 1 to 5% by weight of processing aids and granulated again using heat treatment in suitable granulating apparatus. The granules obtained are non-staining, free flowing and retain original colour and crystallinity of curcuminoids. Thus these are suitable granular and non-staining compositions for formulating in variety of dosage forms of curcuminoids

The granules may be formed by varying the heat treatment in the range of 50 to 200° C., using suitable granulation process such as hot melt granulation or hot melt extrusion. The resulting granular composition is significantly non-staining to equipment and contact surfaces and it is suitable for converting into finished dosage forms such as tablets, capsules, caplets, beads, beadlets and the like.

The composition as described herein may overcome process difficulties such as bulk handling of active unformulated curcuminoids due to the colour explosion and dusting characteristics. The routine problem of staining due to curcuminoids, makes the production process troublesome, expensive and time consuming because of complete change over cleaning required using organic solvents after processing the batches manufacturing. The composition as described herein helps to overcome these difficulties. The composition as described herein provides high content of non-staining curcuminoids in granular form, which is convenient to process in finished dosage form, thus providing clean and cost-effective alternative to handling regular staining curcuminoids in powder form.

The composition as described herein is prepared by solvent-free process and it is in the form of granules, which exhibit non-staining property. Curcuminoid granules prepared by this process overcome the tedious problems of staining of the equipment and other contact surfaces and therefore are useful for formulation into finished dosage forms. Non-staining curcuminoid granules as described herein prevent problem of dusting of curcuminoids in and around formulation area.

The non-staining granular compositions as described herein have various advantages with respect to industrial processing. The composition is comprised of high content of curcuminoids by weight of granules as the composition is substantially free of the excipients. Curcuminoid granules is a non-staining composition and can be formulated into tablets, capsules and other dosage forms by using suitable processes and equipment employed in nutraceutical, pharmaceutical or food industry. The granules do not stain the equipment used in the processing, thus making the formulation process convenient and industrially useful.

According to one more embodiment, curcuminoid granules may be converted in tablet dosage forms by adding excipients selected from the group of, but not restricted to diluents, fillers, carriers, stabilizers, antioxidants, anti-tacking agents, glidants, disintegrants, lubricants, oil vehicles and the like. The composition, being non-staining in nature, will not stain the equipment involved in formulation processes, thus making the process non-tedious, convenient and industrially useful.

The non-staining curcuminoid composition, as described herein is granular in nature, retains original yellowish orange colour and crystalline form of unformulated curcuminoids. The composition is substantially free of any excipients and thus may be comprised of very high content of active, at least about 94% curcuminoids in the form of free-flowing granules. The invention also relates to process for preparation of non-staining curcuminoid granules, without use of any excipient. The process described herein is solvent free, wherein the active may be subjected to one step continuous granulation method. Although the process results in maintaining yellowish orange natural colour of curcuminoids, these granules do not cause staining and dusting of curcuminoids during formulating into finished dosage forms. The non-staining curcuminoid compositions are thus industrially useful and suitable for various formulation related operations, without any colour explosion.

The granular composition of curcuminoids can be evaluated through characterization of the granules for properties like non-staining property, assay content, crystallinity along with formulation stability.

### EXAMPLES

### Example 1: Non-Staining Curcuminoid Composition Comprising 0 to 5% Wax

TABLE 1
A. Composition of Formula 1 to 6 (with and without processing aid)

| | Formula | Formula | Formula | Formula | Formula | Formula |
|---|---|---|---|---|---|---|
| Ingredients | 1 | 2 | 3 | 4 | 5 | 6 |
| Curcuminoids 95% | 100.0% | 99.0% | 98.0% | 97.0% | 96.0% | 95.0% |
| Carnauba wax | 0.0 | 1.0% | 2.0% | 3.0% | 4.0% | 5.0% |
| Total | 100.0% | 100.0% | 100.0% | 100.0% | 100.0% | 100.0% |

### Process for Preparation of Formula 1

Weighed quantity of extract comprising curcuminoids is melt granulated in Hot Melt Extruder operating with the heating range of 150-180° C. in various compartments. The resultant granules are sized to get the granules in the range of 150 to 800 microns.

### Process for Preparation of Formula 2 to Formula 6

Weighed quantity of curcuminoids are melt granulated in Hot Melt Extruder operating with the heating range of 150-180° C. in various compartments. The resultant granules are mixed with corresponding quantity of carnauba wax and again melt granulated in Hot Melt Extruder operating with the heating range of 50-80° C. in various compartments. The re-granulated non-staining composition is sized to have granules varying in size from 200 to 600 microns.

B. Determination of Curcuminoids Content

Non-staining composition is analyzed for its total curcuminoids content by using HPLC (High Performance Liquid Chromatography). Weighed sample equivalent to 50 mg of Curcuminoids is powdered and transferred into 200 mL dry volumetric flask, to which methanol is added and subjected to centrifugation. 2 mL of the supernatant solution was transferred to 100 mL volumetric flask and the volume is made-up with mobile phase (Tetrahydrofuran: Buffer (40:60). The solution is filtered through 0.45 μm PVDF filter. The detection is done by using C18 column at 420 nm, using flow rate as 1.0 mL/min and 100 μL Injection Volume. Total curcuminoids content is calculated using the area of test solutions and standard solution

TABLE 2
Determination of curcuminoid content of Formula 1 to 6

| | | Formula | Formula | Formula | Formula | Formula | Formula |
|---|---|---|---|---|---|---|---|
| Assay | Active | 1 | 2 | 3 | 4 | 5 | 6 |
| Total | 103.2% | 100.0% | 100.4% | 98.9% | 98.8% | 97.9% | 98.0% |
| curcuminoids | | | | | | | |

The active content of the compositions is observed to be in the range of 97 to 100.5%.

C. Evaluation of Non-Staining Properties of the Composition

Curcuminoids granular composition is analyzed for non-staining property by the following method:
a) 500 mg of sample is weighed on butter paper and it is transferred and spread on 10×10 cm² stainless steel plate. The plate is then tilted so that powder falls on butter paper. The plate is observed carefully to check whether the sample sticks to the plate.
b) The plate is rinsed using 10 ml of 90% methanol in water and the rinsate is collected in 25 ml glass beaker. This procedure is repeated for 3 times and each time the rinsate is collected in separate glass beaker and absorbance is checked at 420 nm against standard solution; which is prepared by weighing 50 mg of Curcuminoid working standard and dissolved in 90% methanol in water.

TABLE 3
Evaluation of non-staining property of Formula 1 to 6

| Assay | | Formula | Formula | Formula | Formula | Formula | Formula |
|---|---|---|---|---|---|---|---|
| (mg) | Active | 1 | 2 | 3 | 4 | 5 | 6 |
| Rinse | 0.32 | 0.28 | 0.25 | 0.09 | 0.05 | 0.05 | 0.04 |
| solution 1 | | | | | | | |
| Rinse | 0.26 | 0.003 | 0.003 | 0.005 | 0.002 | 0.003 | 0.001 |
| solution 2 | | | | | | | |

It can be seen from Table 3 that the compositions comprising 3 to 5% excipient (Formula 4, 5 and 6) did not produce significant staining on the stainless steel plate after first rinsing.

After second rinsing, the composition without excipients (i.e., 0% excipients—Formula 1) did not show any stain on the stainless-steel plate and addition of excipients, at the level of 1-5% as processing aid, did not change the property of non-staining produced by melt granulation process.

If plain active (curcuminoids) is used for preparing final formulations, about 3-4 times cleaning of machine and equipment will be required; whereas the above non-staining compositions will require only 1-2 times cleaning of the equipment, thus saving the time and cost of the processing.

D. Evaluation of Crystallinity of Non-Staining Composition

Non-staining composition of Formula 1 was subjected to XRD analysis and the diffractogram was compared with that of plain curcuminoid to check crystallinity pattern of both the test samples.

It was found that the diffractogram of curcuminoids and the non-staining composition was substantially same, suggesting that the crystallinity pattern of curcuminoids was retained in non-staining composition.

E. Evaluation of Stability of Non-Staining Composition

Composition of Formula 3 was subjected to stability study at different storage conditions as shown in Table 4 and the active content was analyzed over a period of 3 months.

TABLE 4
Stability Data of non-staining curcuminoid composition

| | Stability | Incubation | |
|---|---|---|---|
| Composition | Condition | Period | Assay |
| Formula 3 | Before stability | Initial | 99.3% |
| | 40° C./75% RH | 1 month | 100.5 |
| | | 2 month | 100.3 |
| | | 3 month | 100.2 |
| | 30° C./75% RH | 3 month | 99.2 |
| | 25° C./60% RH | 3 month | 100.5 |

The composition was found to be stable over a period of 3 months at all three different storage conditions.

### Example 2: Non-Staining Curcuminoid Composition Comprising 1 to 5% Cetyl Alcohol

TABLE 5
Composition and active content of non-staining granules
(Formula 7 to 11)

| | Formula | Formula | Formula | Formula | Formula |
|---|---|---|---|---|---|
| Ingredients | 7 | 8 | 9 | 10 | 11 |
| Curcuminoids | 99.0% | 98.0% | 97.0% | 96.0% | 95.0% |
| 95% | | | | | |
| Cetyl alcohol | 1.0% | 2.0% | 3.0% | 4.0% | 5.0% |
| Total | 100.0% | 100.0% | 100.0% | 100.0% | 100.0% |

Assay

| | | | | | |
|---|---|---|---|---|---|
| Total | 101.8% | 101.3% | 99.9% | 99.8% | 98.0% |
| curcuminoids | | | | | |

Process: Weighed quantity of curcuminoids are melt granulated in Hot Melt Extruder operating with the heating range of 150-180° C. in various compartments. The resultant granules are mixed with corresponding quantity of cetyl alcohol and again melt granulated in Hot Melt Extruder operating with the heating range of 50-80° C. in various compartments. The re-granulated non-staining composition is sized to have granules varying in size from 200 to 600 microns.

The composition is evaluated for active content and non-staining property.

TABLE 6
Evaluation of Non-staining properties of the composition

| Assay | | Formula | Formula | Formula | Formula | Formula |
|---|---|---|---|---|---|---|
| (mg) | Active | 7 | 8 | 9 | 10 | 11 |
| Rinse | 0.32 | 0.24 | 0.25 | 0.14 | 0.19 | 0.15 |
| solution 1 | | | | | | |
| Rinse | 0.26 | 0.005 | 0.004 | 0.0004 | 0.0009 | 0.0003 |
| solution 2 | | | | | | |
| Rinse | 0.03 | 0.0007 | 0.0001 | 0.0001 | 0.0006 | 0.0001 |
| solution 3 | | | | | | |

The formulations contain excipient at the levels of 1 to 5% did not produce stain on the stainless-steel plate, even after second rinsing.

If the unformulated active is used in the formulation, there will be need of 3-4 times cleaning of machine and equipment whereas the above said non-staining compositions will require 1-2 times cleaning, thus resulting into saving of time and cost in formulating the finished dosage forms using non-staining granular curcuminoid compositions.

### Example 3: Non-Staining Curcuminoid Composition Comprising 1 to 3% Glyceryl Behenate

TABLE 7
Composition and active content of non-staining granules
(Formula 12 to 14)

| Ingredients | Formula 12 | Formula 13 | Formula 14 |
|---|---|---|---|
| Curcuminoids 95% | 99.0% | 98.0% | 97.0% |
| Glyceryl Behenate | 1.0% | 2.0% | 3.0% |
| Total | 100.0% | 100.0% | 100.0% |

Assay

| | | | |
|---|---|---|---|
| Total curcuminoids | 100.7% | 99.9% | 99.8% |

Process: Weighed quantity of extract comprising curcuminoids is melt granulated in Hot Melt Extruder operating with the heating range of 150-180° C. in various compartments. The resultant granules are mixed with corresponding quantity of glyceryl behenate and again melt granulated in Hot Melt Extruder operating with the heating range of 50-80° C. in various compartments. The re-granulated non-staining composition is sized to have granules varying in size from 200 to 600 microns. The composition is evaluated for active content and non-staining property.

TABLE 8
Evaluation of non-staining properties of Formula 12 to 14

| | | With 1% | With 2% | With 3% |
|---|---|---|---|---|
| Assay (mg) | Active | excipients | excipients | excipients |
| Rinse solution 1 | 0.32 | 0.27 | 0.25 | 0.20 |
| Rinse solution 2 | 0.26 | 0.001 | 0.002 | 0.004 |

The compositions comprising excipient at levels of 1 to 3% did not produce stain on the stainless-steel plate after second rinsing, indicating non-staining property of curcuminoid granular compositions on equipment or contact surfaces.

1. A non-staining composition comprising high content of curcuminoids in granular form; wherein the composition is substantially free of excipients.

2. The non-staining composition of claim 1, wherein curcuminoid granules do not cause staining and dusting of equipment and contact surfaces while formulating into finished dosage forms.

3. The non-staining composition of claim 1, which may be comprised of at least about 94% of curcuminoids and about 0 to 5% by weight of excipients, which are used as processing aid in nutraceutical, food and cosmetics industry.

4. The non-staining composition of claim 3, wherein the processing aid may be selected from the class of lipids, fats, fatty alcohol, fatty acid esters, waxes, oils, their derivatives and the combination thereof.

5. The non-staining composition of claim 4, wherein the processing aid is selected from carnauba wax, beeswax, stearic acid, cetyl alcohol, pegylated fatty acids, glycerol fatty acid esters, monoglycerides, diglycerides, triglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, glycolipids, glycerolipid, sphingolipid, phosphatides, phosphatic acid, phosphatidylethanolamine, phosphatidylcholine, phosphatidylserine, phosphatidylinositol, glycerophospholipids, cerebrosides, gangliosides, cephalins, sulfatides, sugar esters, sugar ethers, sucrose esters, sterols, polyglycerol esters, ceramide, sterol, fat-soluble vitamins, prenol, saccharolipid, polyketides, their derivatives or the combination thereof.

6. The non-staining composition of claim 1, wherein the granules may vary in size from 50 to 1500 microns, preferably from 100 to 1000 microns.

7. The solvent free process for preparation of non-staining composition with high content of curcuminoids, which is comprised of
a. subjecting the curcuminoids to heat treatment using suitable granulator
b. optionally mixing the granules with 0 to 5% by weight of processing aid
c. processing the mixture of step b using heat treatment in suitable granulator to get the granules
d. sizing the resulting granules to get non-staining composition with at least 94% curcuminoids.

8. The process of claim 7, wherein the non-staining composition can be granulated using heat treatment between the temperature ranging from 50 to 200° C., using granulator selected from rapid mixer granulator, extrusion spheronizer, hot melt extruder, roller compactor, fluid bed granulator and the like.

9. The process of claim 7, wherein the processing aid may be selected from fats, lipids, fatty acids, fatty alcohol, waxes, oils and the combination thereof.

10. The non-staining composition of claim 1, which retains original yellowish orange colour of curcuminoids and can be formulated in capsules, tablets, caplets, pills, beads, beadlets, pellets and dosage forms, without staining the equipment and contact surfaces during formulation.
